(19) United States
(12) Patent Application Publication
(10) Pub. No.: US 2022/0241055 A1
(43) Pub. Date: Aug. 4, 2022

(54) DENTAL IMPLANT WITH ANTIBACTERIAL/ ANTIMICROBIAL PROPERTY HAVING METAL-ORGANIC FRAMEWORK (MOF) STRUCTURE AND PRODUCTION METHOD THEREOF

(71) Applicants: Zeynep YESIL DUYMUS, ERZURUM (TR); Vagif NEVRUZOGLU, RIZE (TR); Selcuk DEMIR, Rize (TR); Murat TOMAKIN, Rize (TR); Sabit Melih ATES, Rize (TR); Ipek CAGLAR, Rize (TR); Fatih Saban BERIS, Rize (TR)

(72) Inventors: Zeynep YESIL DUYMUS, ERZURUM (TR); Vagif NEVRUZOGLU, RIZE (TR); Selcuk DEMIR, Rize (TR); Murat TOMAKIN, Rize (TR); Sabit Melih ATES, Rize (TR); Ipek CAGLAR, Rize (TR); Fatih Saban BERIS, Rize (TR)

(73) Assignee: RECEP TAYYIP ERDOGAN UNIVERSITESI, RIZE (TR)

(21) Appl. No.: 17/622,787

(22) PCT Filed: Dec. 25, 2020
(86) PCT No.: PCT/TR2020/051391

§ 371 Date: Dec. 24, 2021

(30) Foreign Application Priority Data
Dec. 31, 2019 (TR) 2019 23108
Aug. 27, 2020 (TR) 2020 13519
Nov. 19, 2020 (TR) 2020 18630

(51) Int. Cl. A61C 8/00; A61L 27/30; A61L 27/54; A61L 27/04

(52) U.S. Cl. CPC A61C 8/0013; A61L 27/306; A61L 27/54; A61L 27/047; A61L 2420/02; A61L 2430/12; A61L 2300/104; A61L 2300/404; A61C 2008/0046

(57) ABSTRACT

The present invention relates to a dental implant that contains a metal-organic framework (MOF) structure involving antibacterial metal ions, especially silver (Ag), thus can release long-term and controlled antibacterial metal ions and exhibits a long-term antibacterial/antimicrobial effect, has osseointegration capability and high biocompatibility and a method for preparing this dental implant.

### FIELD OF THE INVENTION

The present invention relates to a dental implant that contains a metal-organic framework (MOF) structure involving antibacterial metal ions, particularly silver (Ag), thus can release long-term and controlled antibacterial metal ions and exhibits a long-term antibacterial/antimicrobial effect, has osseointegration capability and high biocompatibility and relates to a method for preparing this dental implant.

The invention particularly relates to antibacterial implants with osseointegration ability and high biocompatibility, in the first embodiment of which, the metal-organic framework (MOF) structure containing drugs, antibiotics and/or antibacterial agents, metal ions, preferably silver, is loaded into the cavity formed within the implant and this area is evaluated as an antibacterial/antimicrobial depot, in the second embodiment of which, the adverse effects of vanadium (V) on human health were eliminated by preventing the release of vanadium (V) ions from the basic implant layer of implants made of Ti-6Al-4V alloy, and long-term and controlled drug, antibiotic and/or antibacterial release from the implant to the tissue is provided, and implant bone fusion is improved.

### STATE OF THE ART

A dental implant is a surgical component that forms an interface with the jaw or skull bone so as to support a dental prosthesis such as a crown, bridge, prosthesis, facial prosthesis, or so as to act as an orthodontic anchor. The modern dental implants are based on a biological process called osseointegration, in which materials such as titanium form a closer bond to the bone. First of all, the implant fixture is placed in a manner such that it is likely to osseointegrate, then a dental prosthesis is added. A variable amount of recovery time is required for osseointegration prior to a dental prosthesis is attached to the implant (a tooth, bridge or prosthesis) or an abutment to hold a dental prosthesis/crown is placed.

Osseointegration is realized from six weeks to six months, depending on the quality of the implants, the location of the implants and the health of the jawbone. Temporary teeth can be placed during this period, and when the implants are fully integrated, permanent replacement teeth are fixed on the implants or implant posts. The success or failure of dental implants is directly associated with the general health of the person to whom they are placed, the use of drugs that affects osseointegration, and the health of the tissues within the mouth. Osseointegration, which is also defined as direct contact between the implant and osteoblasts in the host living tissue and the formation of new bone tissue on the implant surface or direct structural and functional integration between the implant surface and living bone without a fibrous tissue (Branemark et al., 2001); not only depends on the recovery capacity of the host bone but also the materials used and reactions caused by the material are important. The stress on the implant and on the prosthesis located on the implant shall also be analyzed for the success of bone integration. Since the biomechanical forces that occur during chewing are effective in determining the location and number of implants in the bone where they will be placed, this fixation is the key to the long-term prosthesis health. Furthermore, the success of dental implants depends on many factors, such as from the surface properties of the applied implants to the method of the practitioner. The most common problems seen with respect to implant materials placed in living tissues are the infections that occur in relation to implant. Infection may occur in the implant area in cases such as not working under sufficiently sterile conditions, not placing it at the right angle in the jawbone, not well-fitting the prosthesis on the implant, smoking, and ignoring the periodic maintenance of the teeth. The formation of a successful implant-bone fusion and its sustainability in the long term is critical so as to prevent the occurrence of infection, (Liu et al., 2016). In studies carried out in the state of the art, it has been mentioned that infections that occur in relation to the implant can be prevented by preventing the bacterial adhesion that may occur on the implant surface from the first moment the implant is placed in the host tissue (Tamilvanan et al., 2008). Therefore, different methods such as loading antibiotic-based drugs have been used to give implant materials antibacterial properties, but the long-term effectiveness of these methods was not found to be sufficient (Liu et al., 2016, Neut et al., 2003).

Today, sandblasting and acid roughening methods are preferred as general surface properties in many implant systems (Scacchi et al., 2000). In some studies, it has been mentioned that sandblasting provides osteoblast adhesion, proliferation and differentiation (Miao et al., 2017, Pramano et al., 2016). On the other hand, it was observed that fibroblasts have more difficulty in bonding to these surfaces, which may limit soft tissue proliferation and potentially benefit bone formation (Abron et al., 2001). It is suggested to roughen the titanium substructure with acid so as to remove the residues that remain after sandblasting from the implant surface (Pramano et al., 2016). Acid etching can reduce the mechanical properties of titanium. Titanium forming micro cracks on the implant surface that can reduce the fatigue strength may cause the elimination of hydrogen embrittlement (becoming brittle, softening) (Yokoyama et al., 2002).

Today, when different commercial implant systems are considered, it is seen that different sandblasting and etching processes are applied. Some of the applicable surface treatments are in the form of coating the implant surface with insulin or bisphosphonates or bioactive agents and making the implant surfaces hydrophilic (Avila et al., 2009, Marenzi et al., 2019).

The implant system for bone, which is widely used in the state of the art consists of three parts. These are as follows;
a) Infrastructure: It is the substructure of the implant structure and expresses the implant screw (fixture) and post (abutment) that is surgically placed in the jawbone.
b) Suprastructure: It is the intermediate structure of the implant system, it refers to the prosthesis made on the implant.
c) Mesostructure: It is the intermediate structure of the implant system. The mesostructure part, which is defined as the supporting screw, ensures to combine the infrastructure and the suprastructure and is involved in the overdenture type prosthesis made on the implants.

In the application of the mesostructure part combining the infrastructure and suprastructure parts, it does not go to the end of the screwed channel and when the combining process is completed, a cavity occurs within the infrastructure of the implant. The cavity area formed in the implants used today is approximately 5-7 mm². Depending on the geometric size of this cavity, obtained static pressure values vary between 600-1000 kPa. Since “metal fatigue” occurs in a metal under high pressure over time, the bonds in the metal weaken and structural conversions occur with the effect of high pressure. These structural conversions can convert the metallic implant that is in a biologically active environment into a bacterial settlement site over time. Ensuring a contact between the cavity formed in this period and the tissue enables the implant in the biologically active environment to remain in a stable structure for a long period of time.

Metal-organic framework (MOF) structures are three-dimensional, micro porous and organic-inorganic hybrid materials with high surface area and is a new material class that has been studied extensively in recent years (Furukawa et al., 2013). These materials are formed by transferring metal ions or metal sets with appropriate organic molecules (L) into three-dimension. Using the pores they have for different purposes, their resistance to various chemical and physical processes due to their stable structure and the customization of their usage areas by adding different functional groups to their structures increase the usage areas of these materials. Some of the usage areas of these framework structures can be listed as follows; carbon dioxide, hydrogen etc. gas storage, purification, separation of gases and liquids, shape and size selective catalysis and biomedical applications.

In the biomedical applications of metal-organic framework structures, it is focused on adapting some special reagents to the framework structures and studying their properties; these materials are considered to be useful selecting specific targets (tumors, etc.), reducing the drug dosage required for treatment, extending the duration of drug intake, optimizing pharmacokinetics, and reducing drug side effects (Huxford et al., 2010). As an example, in the state of the art, ibuprofen is loaded into different porous materials and the release rates of ibuprofen that is loaded under conditions similar to body medium (SBF, 37° C.) were determined. According to a performed study, the most amount of ibuprofen can be stored in metal-organic framework structures and the stored drug was released for 6 days (Horcajada et al., 2012). In the literature, there are also some studies in terms of the release of metal ions (silver, etc.) stored in framework structures into the environment. Mortada et al have stored (respectively UiO-67 (bpy) and (UiO-66-2COOAg) silver (Ag) ions in the zirconium-framework structures that they have prepared by using [2,2′-bipyridine]-5,5′-dicarboxylic acid and 1,2,4,5-benzenetetracarboxylic acid and determined that prepared MOF-Ag structures exhibits antibacterial properties against E. Coli (Mortada et al. 2017).

As is known, biological systems are not in balance, and the processes occurring in these systems include irreversible processes. Accordingly, mathematical models applied to biological systems must be based on nonlinear models. One of the signal transmission mechanisms in the living organism is the spread of stimulation waves. Stimulation waves can spread not only through nerve cells but also through skeletal muscles, intestines, blood vessels and heart cells. Researches have shown that the nonlinear partial differential equation given in Equation 1 is valid in clarifying the behavioral types of the biological active systems:

$$\frac{\partial {x}_{i}}{\partial t} = {ff}_{i} ( {x}_{1} , {x}_{2} , … , {x}_{n} ) + \frac{\partial }{\partial r} ( {\sum }_{j = 1}^{n} {D}_{ij} \frac{\partial {x}_{j}}{\partial r} ) , i = 1 , 2 , … , n \qquad ( 1 )$$

Here, the diffusion coefficient Dᵢ, the mutual diffusion coefficient of Dᵢⱼ, is a nonlinear function fᵢ expressing the interaction between compounds in the substance. A living cell volume has a liquid environment for the occurrence of active diffusion process. Furthermore, although molecules move very slowly in vivo, cell membranes that consist of lipids facilitate the active diffusion of the molecules. The transfer of ions and macromolecules through the membranes occurs through specific mechanisms (carriers, channels, etc.). If a substance in vivo contains several components, each component moves in the direction of low concentrations and thus this results in a homogeneous distribution in the environment. This kind of a system converts into a stable status which is called homogeneous. The interactions between components occur as well as diffusion processes occur in the biologically active environment. Interactions that occur between components are defined by nonlinear functions.

In the patent application numbered KR2009014027A in the state of the art; implant fixtures on which a layer of silver or silver alloy with nano or micro thickness is coated on its surface is disclosed. Thus, it is aimed to prevent disadvantages such as implant removal or inflammation etc. In the patent application numbered US20190269484A1; zirconium, ceramic or composite-based implants produced using different techniques and different ions (Ag, Au, Ti, Zr) or particles (hydroxyapatite, tricalcium phosphate, carbon and carbon nanotubes etc.) to improve implant-bone fusion to the surface of these implants is disclosed for dental restorations and surgical bone fixation.

The superiority of titanium (Ti) in resisting corrosion or toxicity in tissues in dental implants, not causing allergic reactions and mechanical strength made titanium the most preferred metal in dentistry. The biocompatibility of titanium basically depends on its ability to form an impermeable layer of titanium oxide (TiO₂) that acts as a barrier against chemical attacks due to the fluids in the human body, as well as its low electrical conductivity. Commercially pure (CP) titanium has the highest resistance to corrosion and is generally accepted as the most biocompatible metal due to its stable/inert oxide layer. However, commercially pure (CP) titanium is a material that is extremely costly compared to other metals, since it is rare and difficult to process. Therefore, dental implants made of commercially pure (CP) titanium cause high costs due to the nature of titanium.

In the state of the art American patent numbered “U.S. Pat. No. 9,095,391B2”, new orthopedic bone screws/spinal pedicle screws/implants containing coatings to assist in forming a structurally stable interface between the patient's bone/tissue and methods of coating such screws and implants are disclosed. These implants and methodologies include at least one coating, which facilitates osseointegration in patients with infection but requires stabilization or requires coatings that prevent galvanic corrosive reactions and also reduce the risk of infection in immunocompromised patients. Titanium wire and fiber coatings can be used to adhere to Ti-6AL-4V by means of sintering bonding process at low temperature in these implants. Among the alternative methods, spraying a commercially pure CP titanium metal on the Ti-6AL-4V implant surface in the form of plasma is found. However, there are studies which show the fact that titanium plasma spray and hydroxyapatite coatings are separated from the implant surface. In this patent in the state of the art, rather than producing the implant completely from commercially pure titanium, a more economical alternative method is proposed, since the amount of titanium in the implant is reduced by using titanium alloy (Ti-6AL-4V). However, in Ti-6AL-4V alloy, vanadium (V, 4%) element is used as a hardening additive so as to improve the mechanical properties of the implants. The presence of vanadium (V) has potentially toxic effects on cells and living tissues in the human body. It is known that the release of vanadium (V) ions from alloys by passive dissolution can cause color change in the surrounding tissue or lead to an inflammatory response that can cause pain and even relaxion due to osteolysis. In this sense, it is important to develop coatings to be applied on the implant surface so as to prevent the mobility of vanadium (V) ions to the human body. Since the vanadium ions in these implants release to the tissue, thus Ti-6Al-4V alloy becomes prominent as a material in literature which has a toxic effect for the body and whose biocompatibility is questioned.

In the state of the art, an implant that contains a bioactive substance and an antimicrobial agent is disclosed in the Japanese patent application numbered “JP2015057126A”. This implant has at least one coating surface that contains a bioactive agent and an antimicrobial agent, wherein the concentration of the antimicrobial agent varies according to the distance from the coating interface. The substance of the implant with antimicrobial feature can be silver, copper, zinc or any combination thereof. However, the release of metal with antibacterial effect is transient in the implants that contain coatings with metal ions such as silver so as to gain the mentioned antibacterial property. That is to say, the effectiveness of silver, copper etc. is very short and the expected antibacterial activity cannot be provided in the long term. At the same time, the uncontrolled, short-term and sudden release of metallic ions from the implant surface causes serious health problems for the human body by creating a toxic effect.

A development is required to be made in the relevant technical field due to not evaluating the cavity region within the implant in current dental implants, high cost of using commercially pure titanium, toxic effect of vanadium (V, 4%) used as a hardening additive in Ti-6Al-4V alloy developed to reduce the cost of implants on cells and living tissues in the human body, difficulties and insufficiencies of antibacterial efficiency of implants with antibacterial properties such as silver and copper for short-term, uncontrolled and toxic effects.

### BRIEF DESCRIPTION OF THE INVENTION AND AIMS OF THE INVENTION

The present invention relates to a dental implant that contains a metal organic framework (MOF) structure involving antibacterial metal ions, especially silver (Ag), thus can release long-term and controlled antibacterial metal ions and exhibits a long-term antibacterial/antimicrobial effect, has osseointegration capability and high biocompatibility and a method for preparing this dental implant.

The most important aim of the invention is to develop a dental implant that can exhibit long-term antibacterial/antimicrobial effect with long-term and controlled silver (Ag) release. Since the inventive dental implant is coated with (MOF/Ag), a metal-organic framework (MOF) structure containing silver (Ag), the silver release continues for 9 days and subsequent days following the placement of the implant. Therefore, the antibacterial/antimicrobial effect can continue during this time. Antibacterial implants are produced that prevent tissue infection after the implant process and ensure more effective treatment.

An aim of the invention is to evaluate the cavity within the implant, which is loaded with the antibacterial agent-added MOF structure, as an antibacterial depot. At the same time, micro channels are created in the implant with laser beams so as to provide the integration of the implant with the tissue.

An aim of the invention, by applying drug, antibiotic and/or antibacterial agent-added MOF to the cavity between the implant abutment screw and the implant, is to connect the micro channels opened in this area and the outer surface of the implant connected with the bone and thus to improve the implant-bone fusion.

Another aim of the invention is to prevent the release of vanadium (V) which is used as a hardening additive, which is the disadvantage of Ti-6Al-4V alloy developed to reduce the cost of implants; is to eliminate the negative effects of vanadium (V) to human health by using Ti-6Al-4V alloy instead of pure titanium (Ti) by both continuing the economic benefit provided with this alloy and preventing the release of vanadium (V) seen as a disadvantage of Ti-6Al-4V alloy. In the inventive implant, it has been determined that there is a sharp decrease between the control sample and a metal-organic framework (MOF) structure containing silver (Ag) in terms of vanadium release and the MOF/Ag layer significantly prevents the release of vanadium (V).

Another aim of the invention is to remove the high static pressure that occurs in the cavity and triggers the structural transformation. The pressure effect is eliminated since the MOF structure is added to the cavity within the implant in the present invention.

Another aim of the invention is to provide a dental implant with high osseointegration capability and biocompatibility. In the PBS solution, it is observed that as a result of the reaction of the inventive MOF/Ag coated Ti-6Al-4V alloy implant with phosphate ions (PO₄³⁻) the surface of the MOF/Ag coated Ti-6Al-4V alloy is smoother. Therefore, the prepared implant can easily interact with PO₄³⁻, which are the main component of the bone, and accelerate bone formation (osseointegration).

The inventive metal-organic framework structure (MOF/Ag) with silver (Ag) content is applicable to all implant types. Furthermore, the metal-organic framework [MOF-(COOH)₂] structure produced in the invention is suitable for loading all metal ions (Au, Cu, Zn, V, Ti, Cr, Co, Ni, Tb, W, Cd, Hg etc.). Similarly, controlled and long-term gold (Au), copper (Cu), zinc (Zn), vanadium (V), titanium (Ti), chromium (Cr), cobalt (Co), nickel (Ni), terbium (Tb), tungsten (W), cadmium (Cd), mercury (Hg) release is also provided.

### DESCRIPTION OF THE FIGURES

In order to understand the advantages of the present invention with its structure and additional elements, it shall be evaluated with the following defined figures.

FIG. 1: Schematic view of the cavity region formed in the implant applications.

FIG. 2: Schematic view of the implant after drilling process for the first embodiment of the invention.

FIG. 3: Plan schematic view of the implant whose drilling process is completed for the first embodiment of the invention.

FIG. 4: In the first embodiment of the invention, X-rays powder diffraction patterns of (A) UiO-66-(Zr)—(COOH)₂ (MOF) and (B) UiO-66-(Zr)—(COOAg)₂ (MOF/Ag) framework structures.

FIG. 5: In the first embodiment of the invention, SEM view of (A) UiO-66-(Zr)—(COOH)₂ (MOF) and (B) UiO-66-(Zr)—(COOAg)₂ (MOF/Ag) framework structures.

FIG. 6: In the first embodiment of the invention, view of adsorption isoterms of (A) UiO-66-(Zr)—(COOH)₂ (MOF) and (B) UiO-66-(Zr)—(COOAg)₂ (MOF/Ag) framework structures.

FIG. 7: EDS graph of UiO-66-(Zr)—(COOAg)₂ (MOF/Ag) framework structure in the first embodiment of the invention.

FIG. 8: In the first embodiment of the invention, the graph of the amount of silver release change in MOF/Ag structure at different temperature values

FIG. 9: Schematic view of one-dimensional tube model of the first embodiment of the invention.

FIG. 10: a) Vanadium, b) Aluminum and c) Silver release graphs of the second embodiment of the invention against time.

FIG. 11: SEM images of the second embodiment of the invention at different magnifications of a) control sample kept in PBS and b) dental implant sample coated with antibacterial (MOF/Ag) kept in PBS.

FIG. 12: EDS images of dental implant coated with antibacterial (MOF/Ag) kept in PBS.

### DESCRIPTION OF THE REFERENCES

In order to better explain the dental implant containing the metal-organic framework structure (MOF) that contains the antibacterial metal ions, especially silver (Ag) developed with the first embodiment of the invention, the parts and components in the figures are enumerated and the corresponding of each number is given below:
1. Implant
2. Implant screw
3. Cavity
4. MOF/Ag structure
5. Infrastructure
6. Suprastructure
7. Mesostructure
8. Bone tissue
9. Channel

### DETAILED DESCRIPTION OF THE INVENTION

The present invention relates to a dental implant that contains a metal-organic framework (MOF) structure involving antibacterial metal ions, especially silver (Ag), thus can release long-term and controlled antibacterial metal ions and exhibits a long-term antibacterial/antimicrobial effect, has osseointegration capability and high biocompatibility and a method for preparing this dental implant. The first and second embodiments of the invention are common in terms of containing metal-organic framework (MOF) structure containing especially silver (Ag) among metal ions with high osseointegration ability and high biocompatibility, which can release long-term and controlled antibacterial metal ions and thus exhibit a long-term antibacterial/antimicrobial effect.

In the course of the description, hereinafter the metal-organic framework (MOF) containing silver (Ag) will be referred to as MOF/Ag.

The first embodiment of the invention relates to antibacterial implants (1) in which, the metal-organic framework (MOF) structure containing drugs, antibiotics and/or antibacterial agents, metal ions, preferably silver, is loaded into the cavity (3) formed within the implant and long-term and controlled drug, antibiotic and/or antibacterial release from the implant to the tissue is provided by evaluating this area as an antibacterial/antimicrobial depot and the implant bone fusion is improved and relates to the production method thereof. In the first embodiment of the invention, the implant loaded with the “metal-organic framework (MOF) structure containing (Ag)” can be made of any material.

The second embodiment of the invention relates to a dental implant that is coated with metal-organic framework (MOF) structures containing antibacterial metal ions, especially silver (Ag), thus can release long-term and controlled antibacterial metal ions and exhibits a long-term antibacterial/antimicrobial effect, eliminates particularly the adverse effects of vanadium (V) on human health by preventing the release of vanadium (V) ions from basic implant layer of implants made of Ti-6Al-4V, has osseointegration capability and high biocompatibility and a method for preparing this dental implant.

Osseointegration is the direct attachment or bonding of bone tissue (8) to an inert, alloplastic material without interfering with the connective tissue, the present invention provides the improvement of the implant's osseointegration ability and implant bone fusion by means of the metal-organic framework (MOF) structure containing silver (Ag). It is critical to improve the osseointegration ability for implant stability and is a prerequisite for implant loading and the long-term clinical success of endosseous dental implants. It also creates a long-term antibacterial/antimicrobial effect by providing long-term and controlled release of silver ions within the structure of MOF/Ag. The silver ions are released in amounts that do not cause toxic effects and exhibit effective antimicrobial/antibacterial properties by means of the long term and controlled release of antibacterial/antimicrobial silver ions.

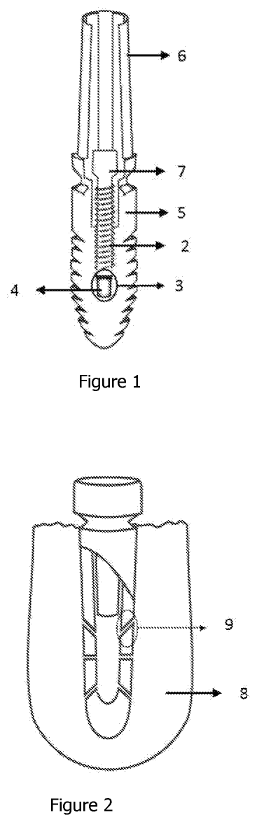

The mesostructure (7) part that combines the infrastructure (5) and suprastructure (6) parts, does not go to the end of the implant screw (2) channel and when the combining process is completed, a cavity (3) occurs within the infrastructure (5) of the implant. (FIG. 1) This cavity (3) is approximately 5-7 mm² and the cavity (3) area can be adjusted. For example; in case the area is required to be enlarged, processes can be performed in terms of increasing the channel depth within the infrastructure (5) or shortening the mesostructure (7) size. The first embodiment of the invention is the antibacterial implants (1) in which, the metal-organic framework (MOF) structure containing metal ions, particularly silver (Ag) is loaded into the cavity (3) formed within the implant (1) structure and long-term and controlled drug, antibiotic and/or antibacterial release is provided from the implant (1) to the bone tissue (8) by evaluating this area as an antibacterial/antimicrobial depot and the implant (1) bone fusion is improved.

In the first embodiment of the invention, 1-20 channels (9) of approximately 100 nm-500 μm are opened in the implant by using laser beams to integrate the cavity (3) within the implant's body with the jaw (bone) tissue (8). The technical features of the laser used in the implant (1) drilling process are preferably as follows:

Wave length λ=800 nm,

Pulse width 90 fs,

Laser beam intensity at the focal point is 1015 W/cm²,

Lens focal distance 5 cm

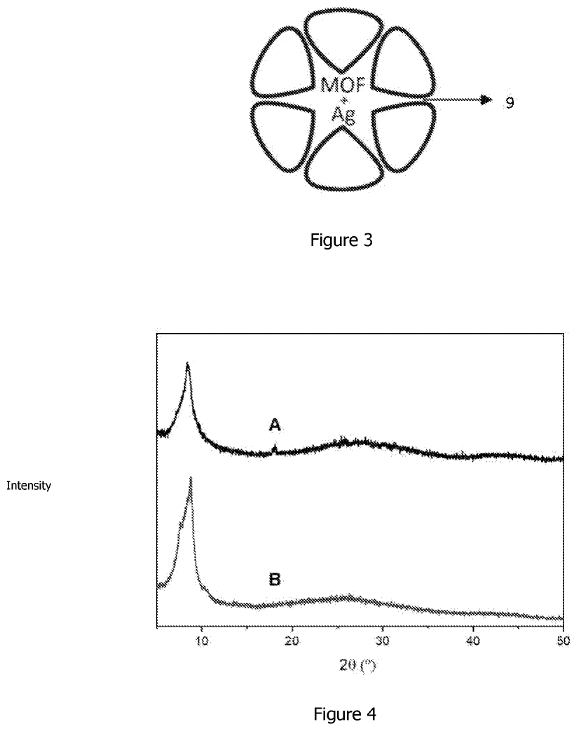

In the first embodiment of the invention, it is required to correspond the direction of the laser beams with the cavity (3) formed in the implant in the drilling process. In FIG. 2, the schematic view of the micro channels (9) created in the implant after drilling is given. The aim of creating holes at different angles is to provide silver ions to move to the tissue at different angles. In FIG. 3, the top view of the implant (1) whose drilling process has been completed is given. It was seen as a result of the measurements that the drilling process did not affect the mechanical properties of the implant. Furthermore, the high static pressure effect on the implant due to the cavity (3) is also removed with this application, thus the structural conversion of the implant with the effect of pressure over time is eliminated.

In the first embodiment of the invention, the chemical preparation of the MOF/Ag structure (4) is performed in two steps. In the first step, while the porous MOF structure is produced; in the second stage, silver (Ag) ions are loaded to the porous MOF structure. Subsequently MOF structures, to which prepared silver ions are loaded, are loaded into the cavity (3) formed in the infrastructure (5) section of the dental implant. As a consequence, antibacterial implants are obtained wherein the cavity (3) region formed in the implant is evaluated as an antibacterial/antimicrobial depot, a drug, and antibiotic and/or antimicrobial agent, preferably silver added metal-organic framework (MOF) structure is loaded into this cavity (3).

In the first embodiment of the invention, the preparation method of the antibacterial dental implant containing the antibacterial MOF/Ag structure (4) comprises the following process steps;
i. Adding a solution containing 100-900 mg 1,2,4,5-benzentetracarboxylic acid (pyromellitic acid) in 1-500 mL of water on a solution containing 100-900 mg of zirconium tetrachloride (ZrCl₄) in 1-500 mL of water and stirring the same for 1-600 min,
ii. Adding 1-50 mL of hydrochloric acid (HCl) (37%, w/w) to each container and keeping the resulting solution for 0.1-96 hours in the incubator at 40-220° C., after dispersing 1-100 mL of the solution into Teflon containers,
iii. Taking the formed framework structure from the incubator at the end of 0.1-96 hours and filtering the same under vacuum and washing the same with hot water,
iv. Heating the framework structure at 30-180° C. for 0.1-48 hours so as to remove the solvent molecules in its pores,
v. Adding 1-1000 mg of framework structure to 1-1000 mL of methanol (MeOH) containing 1-100 mg of silver nitrate (AgNO₃) and mixing the same for 0.1-48 hours at 25-150° C. so as to obtain a MOF/Ag structure (4),
vi. Separating the obtained MOF/Ag structure (4) from the solution and washing the same with methanol (MeOH),
vii. Drying MOF/Ag structure (4) at 30-180° C. under vacuum,
viii. Loading the obtained MOF/Ag structure (4) into the cavity (3) formed in the infrastructure (5) section of the dental implant.

In the process steps of the method in the first embodiment of the above invention, any drug, antimicrobial/antibacterial agent, antibiotic can be loaded instead of silver ion in the process step (v).

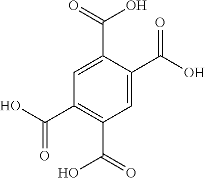 [SMILES: O=C(O)c1cc(C(=O)O)c(C(=O)O)cc1C(=O)O]

### 1,2,4,5-Benzentetracarboxylic acid (pyromellitic acid)

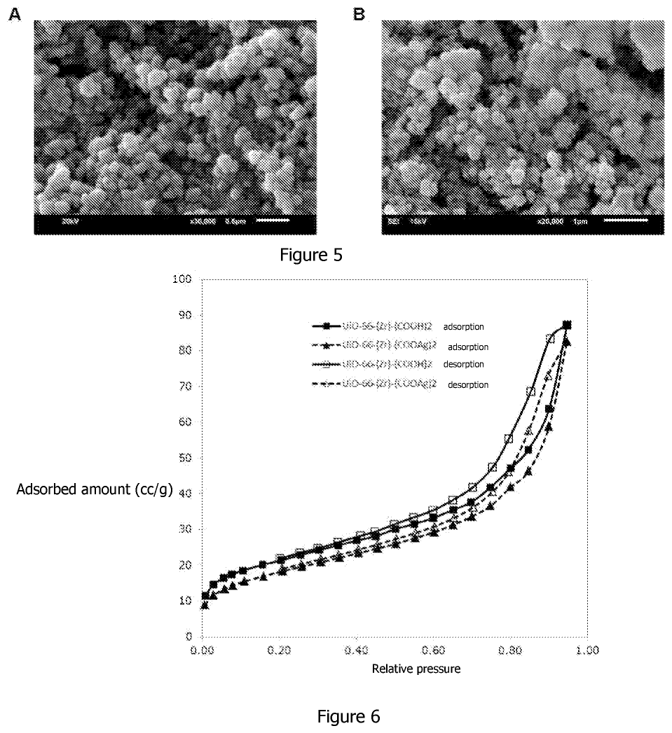

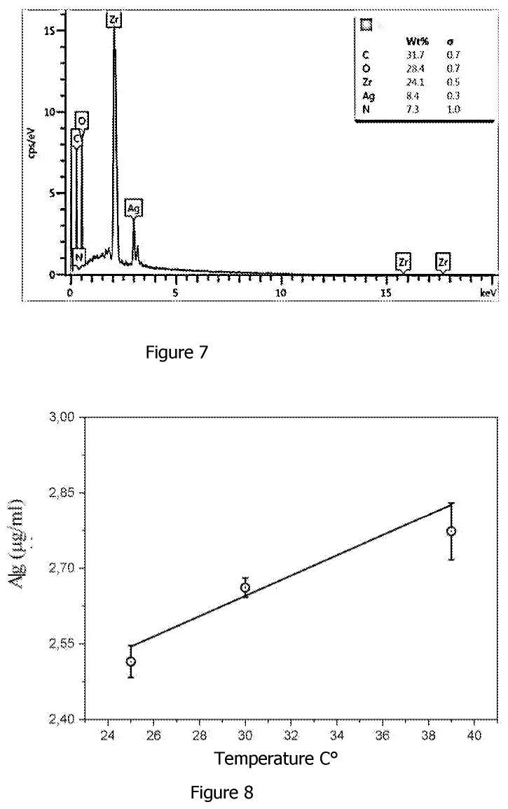

In the first embodiment of the invention, the metal organic framework structure [UiO-66-(Zr)—(COOH)₂] (MOF) having the formulation Zr₆O₄(OH)₄(C₁₀H₄O₈)₄(H₂O)₄(OH)₄, as a consequence of the combination of carboxylate anions formed as a result of deprotonation exposure of used 1,2,4,5-benzenetetracarboxylic acid molecules in reaction environment of two of the carboxylic acids and zirconium metals. While Ag ions react with the carboxylic acid groups of UiO-66-(Zr)—(COOH)₂ framework structure that was not subjected to depronotation, it forms the framework structure [UiO-66-(Zr)—(COOAg)₂] (MOF/Ag) having (Zr₆(O)₄(OH)₄(C₁₀H₂O₈Ag₂)₄(H₂O)₄(OH)₄) formula. As a result of the analyses made, no change was observed in the X-ray powder diffraction (PXRD) patterns (FIG. 4) of the framework structures, confirms the protection of crystal structures. When SEM and EDS views of UiO-66-(Zr)—(COOH)₂ (MOF) and UiO-66-(Zr)—(COOAg)₂ (MOF/Ag) are analyzed (FIGS. 5 and 7), it is seen that metallization is homogenous and no physical mixture is formed. While the BET surface area of the UiO-66-(Zr)—(COOH)₂ (MOF) framework structure is 78 m²/g, it decreases to 65.8 m²/g after metallization with silver (FIG. 6). Since there is no change in PXRD patterns; this decrease in surface area is due to metallization of the framework structure. The obtained experimental results indicate that the MOF/Ag structure (4) has been formed. The mobility of Ag ions from the MOF/Ag structure (4) loaded into the cavity (3) to the tissue is based on the diffusion mechanism that is realized in a biologically active environment. In order to clarify the diffusion process that takes place in an active biological environment, a one-dimensional tube fed from a reactor (tank) filled with a specific substance was analyzed. The diffusion flow of a component is perpendicular to the diffusion direction and proportional to the concentration gradient according to Fick's law.

$$I = - D \frac{\partial C}{\partial r} \qquad ( 2 )$$

Here, I is the dispersion flow of the component of interest in the r axis direction.

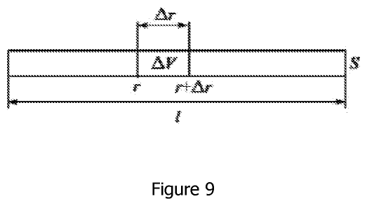

In the case of a one-dimensional tube C (r, t) an equation describing the space-time conversion is derived, the section of the tube considered is S and in this model, ΔVr unit volume with coordinates r and r+Δr is defined. In this case; ΔVᵣ=S·Δr. If we consider that diffusion occurs in the r direction, a schematic view of the one-dimensional tube model is given in FIG. 9. The mass of a substance flowing from the boundary r to the volume Vr in the time interval Δt is equal to the multiplication of the diffusion flow and the S section according to Fick's law.

$$Δ {M}_{r} = - D \frac{\partial C ( r , t )}{\partial r} S Δ t \qquad ( 3 )$$

The ΔMr+Δr mass of the substance that is output from the other boundary zone of the volume with a coordinate of r+Δr is calculated with the equation as follows.

$$Δ {M}_{r + Δ r} = - D \frac{\partial C ( r + Δ r , t )}{\partial r} S Δ t \qquad ( 4 )$$

If in ΔVr volume, it is total mass change; ΔM is calculated with the following equation.

$$Δ M = Δ {M}_{r} - Δ {M}_{r + Δ r} , Δ M = [ D \frac{\partial C ( r + Δ r , t )}{\partial r} - D \frac{\partial C ( r , t )}{\partial r} ] S Δ t \qquad ( 5 )$$

The equation used for the density change is as follows:

$$Δ C = \frac{Δ M}{Δ V} = \frac{Δ M}{S Δ r} = \frac{D \frac{\partial C ( r + Δ r , t )}{\partial r} - D \frac{\partial C ( r , t )}{\partial r}}{Δ r} Δ t \qquad ( 6 )$$

When we pass to Δr→0 limit value, the following equation is obtained.

$$Δ C = \frac{\partial }{\partial r} ( D \frac{\partial C ( r , t )}{\partial r} ) Δ t \qquad ( 7 )$$

When dividing the right and left parts of Equation 7 by Δt and passing to Δt→0 limit value, the following expression is obtained for the differential diffusion equation.

$$\frac{\partial C ( r , t )}{\partial t} = \frac{\partial }{\partial r} ( D \frac{\partial C ( r , t )}{\partial r} ) \qquad ( 8 )$$

If the diffusion coefficient (D) of the medium is constant, the equation is as follows;

$$\frac{\partial C}{\partial t} = D \frac{{\partial }^{2} C}{\partial {r}^{2}} \qquad ( 9 )$$

In case the diffusion process takes place in a three-dimensional space, it converts into equation 10.

$$\frac{\partial C ( r , t )}{\partial t} = D Δ C ( r , t ) \qquad ( 10 )$$

If, in an environment where the diffusion process takes place, there is a substance with densities of Cᵢ (i=1 . . . n) containing n number of different components, then the diffusion equation is as follows.

$$\frac{\partial {C}_{i} ( r , t )}{\partial t} = {D}_{i} Δ {C}_{i} ( r , t ) , i = 1 , 2 , … , n \qquad ( 11 )$$

It is understood that other processes occur in addition to the diffusion process in active biological systems. This causes the addition of new variables expressing the change in density over time to the right section of the equations 9, 10 and 11.

$$\frac{\partial C}{\partial t} = D \frac{{\partial }^{2} C}{\partial {r}^{2}} + F ( r , t ) \qquad ( 12 )$$

Here F is the source function.

It is possible for various conversions to occur as a result of diffusion in multicomponent systems. For example, as the chemical conversions of substances during reactions, it can be considered independent of ffᵢ function, time and place coordinates.

$${ff}_{i} = {ff}_{i} ( {C}_{1} , {C}_{2} , … , {C}_{n} ) \qquad ( 13 )$$

When the chemical conversions that occur at every point of the system are considered, equation 11 can be written as follows.

$$\frac{\partial {C}_{i}}{\partial t} = {ff}_{i} ( {C}_{1} , {C}_{2} , … , {C}_{n} ) + {D}_{i} Δ {C}_{i} ( r , t ) \qquad ( 14 )$$

For an one-dimensional reactor, equation 14 converts to 15.

$$\frac{\partial {C}_{i}}{\partial t} = {ff}_{i} ( {C}_{1} , {C}_{2} , … , {C}_{n} ) + {D}_{i} \frac{{\partial }^{2} {C}_{i}}{\partial {r}^{2}} \qquad ( 15 )$$

The equations 1, 14 and 15 described above are defined as reaction diffusion; this theoretical study is used to calculate the amount of Ag ion release in the antibacterial implant containing the inventive MOF/Ag structure (4) in a biologically active environment.

In the first embodiment of the invention, at the same time, the high static pressure that occurs in the cavity (3) and triggers the structural transformation is eliminated. The pressure effect is eliminated since the MOF structure is added to the cavity (3) within the implant in the present invention.

In the first embodiment of the invention, the amount of silver release was determined experimentally in the implant with MOF/Ag structure (4) content. Since the local temperature of the region increases during the infection process, it is important to determine the amount of Ag release at different temperature values. The amount of silver release increases with the increase of temperature as it is seen in FIG. 8. As can be seen from the diffusion mechanism theory in the active biological environment, the temperature increase in the local infection zone causes the stimulation waves to diffuse stronger by increasing the vibration amplitude of the molecules in this region. Therefore, the releasing amount of the silver ions having weak binding energy in the MOF/Ag structure (4) increases with the increase of temperature. Silver release test was carried out by applying the following process steps:
Adding MOF/Ag for example (2.0 mg) in 5 mL 1×PBS solution (it was prepared by adding 8 g NaCl, 0.2 g KCl, 2.68 g Na₂HPO₄.7H₂O, and 0.24 g KH₂PO₄ into 0.8 Lt distilled water, its volume was completed to 1Lt, pH 7.4),
Placing the samples in the incubator (25, 30, 39° C.) adjusted to different required temperature values and keeping the same in the incubator for a predetermined period (16 hours),
Filtering 0.25 μm syringe through a filter and completing the solution volume to 15 mL,
Determining Ag amount in the solution by ICP analysis.

The experimental results in relation to the silver release test show that the implants containing MOF/Ag produced in the first embodiment of the invention are more effective in antibacterial silver ion release compared to all implant types currently used. The most remarkable point in these results is that the amount of silver release at 39° C., which is defined as the infection temperature, is less than 10 ppm, known as the toxic value. This value shows that the amount of antibacterial silver release is controlled.

The second embodiment of the invention relates to a dental implant that is coated with antibacterial metal ions and with metal-organic framework structures containing especially silver (Ag) among these metal ions, thus can release long-term and controlled antibacterial metal ions and exhibits a long-term antibacterial/antimicrobial effect, eliminates particularly the adverse effects of vanadium (V) on human health by preventing the release of vanadium (V) ions from basic implant layer of implants made of Ti-6Al-4V, has osseointegration capability and high biocompatibility and a method for preparing this dental implant. In the second embodiment of the invention, implants made of Ti-6Al-4V alloy are covered with metal-organic framework structures (MOF) that contain silver (Ag).

In the second embodiment of the invention, first of all the surfaces of implants made of Ti-6Al-4V alloy are prepared for coating, the chemical preparation of the MOF/Ag structure (4) is carried out and in the last stage the coating process is carried out with various coating methods on the implants whose surfaces are prepared.

There are various surface modification methods so as to increase the bioactivity of titanium, a bioinert material. Titanium dioxide (TiO₂) that is formed on the titanium surface plays an important role in the biocompatibility of titanium. When the implant is placed in the human body, the tissue periphery directly contacts with the TiO₂ layer on the titanium surface. Therefore, the biocompatibility of titanium implants depends on the properties of the oxide layer such as surface morphology, chemical composition and surface topography. Particularly, proper implant surface treatment plays an important role in providing successful dental implant applications, such as poor bone quality, immediate loading or other loading procedures etc. The proper progress of the process after the implant is placed in the body and ensuring recovery time earlier are directly related to the surface properties of the implant. Many methods have been developed so as to increase the biocompatibility of titanium-based implants. These are physical/chemical processes and combinations of these methods that only change the surface properties without changing the chemical composition of titanium. While many studies have proved that modification of the topographic structure of the surface increases the biomechanical interaction of the interface with the bone implant contact, in vivo studies show the importance of surface roughness in the development of osseointegration. In addition to this, rough surfaces are preferred for implant applications so as to increase osseointegration rate and osteoblast adhesion. Obtaining higher osseointegration values with increased roughness has been tested in the presence of various coating techniques and a dental implant has been developed that is coated with a metal-organic framework (MOF/Ag) containing silver (Ag), thus can perform long-term and controlled silver (Ag) release, in the light of this information. The second embodiment involves a dental implant that is coated with metal-organic framework (MOF) structures containing silver (Ag), thus can release long-term and controlled antibacterial metal ions and exhibits a long-term antibacterial/antimicrobial effect, eliminates particularly the adverse effects of vanadium (V) on human health by preventing the release of vanadium (V) ions from basic implant layer of implant, has osseointegration capability and high biocompatibility and a method for preparing this dental implant. In the second embodiment of the invention, the preparation method of the dental implant coated with antibacterial MOF/Ag preferably comprises the following process steps,
i. Roughening the surface of implants produced from Ti-6Al-4V alloy under 2-8 bar pressure for 50 seconds by bombarding with Al₂O₃ particles having 300-425 μm dimension,
ii. Oxidizing the surface of rough Ti-6Al-4V implants at 450-700° C. using a tube furnace,
iii. Adding a solution containing 100-900 mg 1,2,4,5-benzentetracarboxylic acid (pyromellitic acid) in 1-500 mL of water on a solution containing 100-900 mg of zirconium tetrachloride (ZrCl₄) in 1-500 mL of water and stirring the same for 1-600 min, iv. Adding 1-50 mL of hydrochloric acid (HCl) to each container
and keeping the resulting solution for 0.1-96 hours in the incubator at 40-220° C., after dispersing 1-100 mL of the solution into Teflon containers,
v. Taking the formed framework structure from the incubator at the end of 0.1-96 hours and filtering the same under vacuum and washing the same with hot water,
vi. Heating the frame structure at 30-180° C. for 0.1-48 hours so as to remove the solvent molecules in its pores,
vii. Adding 1-1000 mg of framework structure to 1-1000 mL of methanol (MeOH) containing 1-100 mg of silver nitrate (AgNO₃) and mixing the same for 0.1-48 hours at 25-150° C. so as to obtain a MOF/Ag structure (4),
viii. Separating the obtained MOF/Ag structure (4) from the solution and washing the same with methanol (MeOH),
ix. Drying MOF/Ag structure (4) at 30-180° C. under vacuum,
x. Adding the dried MOF/Ag structure (4) in 10 ml of ethyl alcohol and preparing the same as a suspension by means of an ultrasonic bath,
xi. Coating the rough Ti-6Al-4V implants whose surface obtained in the process step (ii) is oxidized, with the MOF/Ag structure (4) converted into a suspension and is dried at 80-180° C. temperature by means of using spin coating method, spray coating method and/or dip coating method,
xii. Continuing the coating process until all of the suspended MOF/Ag structure (4) of the process step (xi) is consumed.

In the second embodiment of the invention, the preparation method of the dental implant coated with antibacterial MOF/Ag preferably comprises the following process step,
i. Roughening the surface of implants produced from Ti-6Al-4V alloy under 4 bar pressure for 50 seconds by bombarding with Al₂O₃ particles having 300-425 μm dimension,
ii. Oxidizing the surface of rough Ti-6Al-4V implants at 600° C. using a tube furnace,
iii. Adding a solution containing 100-900 mg 1,2,4,5-benzentetracarboxylic acid (pyromellitic acid) in 1-500 mL of water on a solution containing 100-900 mg of zirconium tetrachloride (ZrCl₄) in 1-500 mL of water and stirring the same for 1-600 min,
iv. Adding 1-50 mL of hydrochloric acid (HCl) (37%, w/w) to each container and keeping the resulting solution for 0.1-96 hours in the incubator at 40-220° C., after dispersing 1-100 mL of the solution into Teflon containers,
v. Taking the formed framework structure from the incubator at the end of 0.1-96 hours and filtering the same under vacuum and washing the same with hot water,
vi. Heating the framework structure at 30-180° C. for 0.1-48 hours so as to remove the solvent molecules in its pores,
vii. Adding 1-1000 mg of framework structure to 1-1000 mL of methanol (MeOH) containing 1-100 mg of silver nitrate (AgNO₃) and mixing the same for 0.1-48 hours at 25-150° C. so as to obtain a MOF/Ag structure (4),
viii. Separating the obtained (MOF/Ag) structure from the solution and washing the same with methanol (MeOH),
ix. Drying MOF/Ag structure at (4) 30-180° C. under vacuum,
x. Adding the dried MOF/Ag structure (4) in 10 ml of ethyl alcohol and preparing the same as a suspension by means of an ultrasonic bath,
xi. (ii) coating the rough Ti-6Al-4V implants whose surface obtained in the process step (ii) is oxidized, with the MOF/Ag structure (4) converted into a suspension and is dried at 140° C. temperature by means of using spin coating method, spray coating method and/or dip coating method,
xii. Continuing the coating process until all of the suspended MOF/Ag structure (4) of the process step (xi) is consumed.

Roughening the surface of implants under 4 bar pressure for 50 seconds by bombarding with Al₂O₃ particles having 300-425 μm dimension and oxidizing the surface at a temperature of 600° C. in the preferred preparation steps of the second embodiment of the invention are discovered by change, these processes are for the formation of TiO₂ in rutile phase. Since TiO₂ layer grows in different phases (rutile, anatase, etc.) and the rutile phase of TiO₂ shall be used for the dental implant to provide the same with a more stable structure. It is observed that the implants made of Ti-6Al-4V alloy coated with antibacterial MOF/Ag obtained with this method in which a more stable rutile phase of dental implants is used, can release long-term and controlled antibacterial metal ions and exhibits a long-term antibacterial/antimicrobial effect, eliminates particularly the adverse effects of vanadium (V) on human health by preventing the release of vanadium (V) ions from basic implant layer of implants, has osseointegration capability and high biocompatibility.

The coating methods used in the second embodiment of the invention, particularly the spin coating method, spray coating method and/or dip coating method were used to improve the quality of the coating. The spin coating method is a process used to produce a thin coating on a hard layer or less inclined substrates (implant). The coating can be divided into 4 stages with the spinning process. These stages are as follows: coating, spinning, spinning termination and evaporation. An amount of liquid is poured on the surface during the implant coating phase. In spinning as the second stage, the fluid flows radially out of the carrier surface of the implant due to the centripetal force. The excess fluid overflows from the carrying surface of the implant and leaves the surface at the end of the rotation. As the film thickness decreases, the amount of liquid that overflows from the surface decreases. The reason for this event can be described with the thinning of the film as the resistance to fluidity increases. At the same time, the increase in the concentration of non-volatile substance causes the resistance to increase against fluidity. The evaporation stage is the final and most important stage in the thinning of coating films. The advantage of the spin coating used in the invention is that the film formed on the surface is evenly distributed while the film is formed on the implant by coating. Hence, the film thickness exhibits a homogeneous property along the surface. The film thickness remains the same unless the MOF/Ag viscosity changes. Two main forces are factors in the homogeneity of film thickness. These forces are the centripetal force and the friction force in the opposite direction that cause the dropped liquid (MOF/Ag) onto the carrier to flow radially outward. The centripetal force during the rotation phase causes the gravitational force to be ignored. Therefore, there is only the centripetal force in the thinning phase of the film. In the invention, the method of dip coating is based on the principle of immersing the implant material used into the solution prepared at a certain speed into MOF/Ag and withdrawing the same at the same speed. The dip coating method takes place in five stages. These phases are as follows: immersing, pulling up, coating, draining and evaporation. As a result of this process, film is formed. In the immersion phase, the implant is immersed in MOF/Ag at a constant speed, while in the pulling up phase; it is pulled up immediately at once. In the coating as the third phase, the sections of the implant that contacts with MOF/Ag are coated. In this phase, gravity force, carrier force between MOF/Ag and implant and surface tension forces are effective. At the end of immersion, the excess MOF/Ag droplets drain from the implant edges and leave the surface, while the MOF/Ag droplets that cannot leave the surface with the draining process evaporate and fly. The MOF/Ag that remains on the implant converts into a film following these steps. The advantage of dip coating is that it is possible to coat substrates (implants) of all shapes and sizes. A proper and controllable thickness can be obtained with this process. Hence, the coated film thickness exhibits a homogeneous property along the surface. The spraying method is spraying the aqueous solutions prepared for the films to be obtained after mixing on the hot base by atomizing them with the help of air or nitrogen gas. Spray coating method is the easiest and cheapest method among thin film obtaining methods. In the invention, usage of spraying method is preferred due to the following reasons, it has a very simple structure, it is more economical in terms of the necessary equipment, it is appropriate for intervention in the production process, a vacuum environment is not required for thin film production and the production process can be followed step by step compared to other methods. Furthermore, this method allows n-type and p-type doping. The quality of the coating varies with experimental parameters such as substrate (implant) temperature, spraying rate and film thickness. In addition to these, experimental parameters such as the diameter of the spray nozzle, the distance of the spray nozzle from the substrate, the ratio of pure water, solution and hydrochloric acid etc. are also important in obtaining a good quality film. The droplet size of the sprayed MOF/Ag has a big impact on the quality of the coating.

In the process step (i) of the second embodiment of the invention, the osseointegration capability of the implant by increasing the biomechanical interaction of the interface (coating) with the bone-implant contact is increased, in the presence of pressure, by bombarding the implants made of Ti-6Al-4V alloy with Al₂O₃ particles, in the process step (ii), a temperature of 600° C. used in the oxidation of the surface of the roughened Ti-6Al-4V implants aims to provide the formation of TiO₂ in the rutile phase. TiO₂ layer grows in different phases (rutile, anatase, etc.) depending on the applied temperature. In order to allow the inventive dental implant to have a more stable structure, the more stable rutile phase of TiO₂ is preferred.

In the second embodiment of the invention, in order to change the silver release amount of the dental implant coated with antibacterial MOF/Ag, depending on the temperature, the following process steps were followed.
i. Placing the dental implant sample coated with MOF/Ag in 5 mL 1×PBS solution (8 g NaCl, 0.2 g KCl, 2.68 g Na₂HPO₄-7H₂O, and 0.24 g KH₂PO₄ were added into 0.8 Lt distilled water, its volume was completed to 1Lt, pH 7.4),
ii. Placing the samples in the incubator that is adjusted to the required temperature (25, 30, 39° C.)
iii. Keeping the same in the incubator for a predetermined period (16 hours).
iv. Filtering 0.25 μm syringe through a filter and completing the solution volume to 15 mL,
v. Determining Ag amount in the solution by ICP analysis.

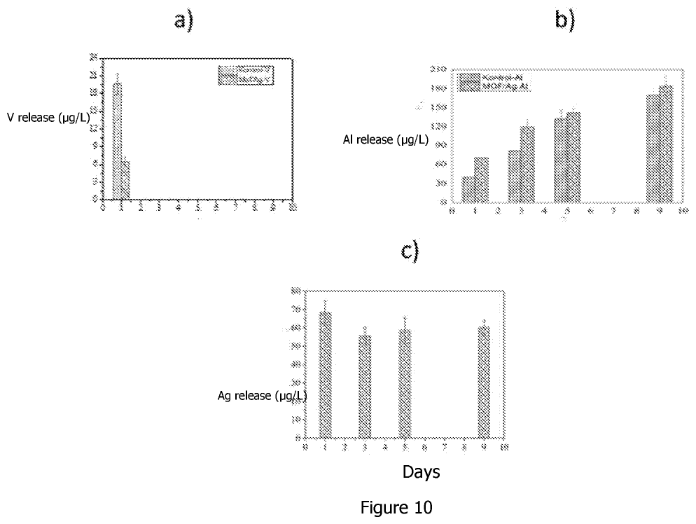

The samples were kept in 1% PBS solution for 9 days and examined by ICP-MS analysis method at regular intervals so as to determine how long the silver (Ag) release continues and the amount of release of other elements in the Ti-6Al-4V alloy. Therefore, the solutions were refreshed every 24 hours with the PBS solution, and control and MOF/Ag coated dental implant samples were collected for 9 days (1st, 3rd, 5th and 9th days). FIG. 10 shows the release graphs of vanadium (V), aluminum (Al) and silver (Ag) against time. The release values for titanium (Ti) could not be determined because it was lower than the sensitivity of the device. It was determined that the silver (Ag) release continued for 9 days and the amount of release did not change significantly according to the ICP results. It was seen that there was a sharp decrease in vanadium release values between the control sample and the dental implant sample coated with antibacterial MOF/Ag and the dental implant coated with antibacterial MOF/Ag significantly prevented vanadium (V) release. In addition to this, V release cannot be determined in the measurements performed for the following days. In the inventive dental implant coated with antibacterial MOF/Ag, the mobility of elements (Ti, Al and V) from Ti-6Al-4V alloy to MOF/Ag structure (4) is performed by diffusion mechanism. Among these elements, Al has the highest diffusion coefficient; (DTi=1.614×10⁻¹⁸ m²/s, DV=1.527×10⁻¹⁸ m²/s, DAl=1, 78×10⁻⁷ m²/s). As it is well known, diffusion of atoms takes place through cavities that exist in the crystalline structure or intermediate regions. However, the diffusion of some elements exhibits amphoteric features. That is to say, both spaces and intermediate regions are used in the diffusion process. For example, diffusion of vanadium in porous SiC crystal structure has amphoteric property. In addition to this, the diffusion mobility can occur faster in porous crystal structures. It is understood that Al atoms in the porous MOF/Ag structure (4) that release faster from the ICP results obtained in this study. Due to the high diffusion coefficient of Al atom and the small ionic radius (rAl³⁺=0.53 Å, rV³⁺=0.64 Å, rAg⁺=1.15 Å), the diffusion process is a situation that is expected to occur more rapidly in the MOF/Ag porous structure. Furthermore, this result shows that the diffusion of Al atoms in the structure of MOF/Ag has amphoteric property. In addition to the low diffusion coefficient of V atoms, the greater atomic radius compared to Al causes their slower movement in the porous structure of MOF/Ag structure (4) and thus the possibility of their settling in the pores increases. In this case, vanadium is placed in the silver (Ag) cavities released from the dental implant coated with the inventive antibacterial MOF/Ag structure (4) and the separation of the settled vanadium (V) from the cavity becomes more difficult due to the low vapor pressure. Therefore, the release of vanadium (V) in the Ti-6Al-4V alloy is prevented. When the situation is examined in terms of hard-soft acid-base theory, since the absolute hardness of silver ions (Ag+6.96) and the absolute hardness of vanadium ions (V³⁺8.70) are close to each other, it is seen that vanadium (V) ions are more likely to be preferred to Ag ions separated from the MOF/Ag framework structure. The dental implant coated with antibacterial MOF/Ag structure (4) prevented vanadium (V) ions from passing into the solution according to the analysis results obtained. Thus, the release of vanadium (V) which is used as a hardening additive, which is the disadvantage of Ti-6Al-4V alloy developed to reduce the cost of implants, is prevented by the invention; while the economic benefit is continued, the negative effects of vanadium on human health are eliminated. In the inventive implant, it has been determined that there is a sharp decrease between the control sample and MOF/Ag structure (4) containing silver (Ag) in terms of vanadium release and the MOF/Ag layer significantly prevents the release of V. However, there is a parallelism between the release values of control and dental implant Al coated with antibacterial MOF/Ag structure (4). Titanium (Ti) and zirconium (Zr) ions, which are possibly found in solutions, were not seen. According to the analysis results in FIG. 10, the amount of silver release (Ag) did not change over time and the silver release continued after 9 days. A dental implant that can exhibit a long-term antibacterial/antimicrobial effect with long-term and controlled silver (Ag) release is developed for 9 days and subsequent days with the invention, the antibacterial/antimicrobial effect continues for 9 days and subsequent days after the implant placement, because it is coated with MOF/Ag, a metal-organic framework (MOF) structure containing silver (Ag).

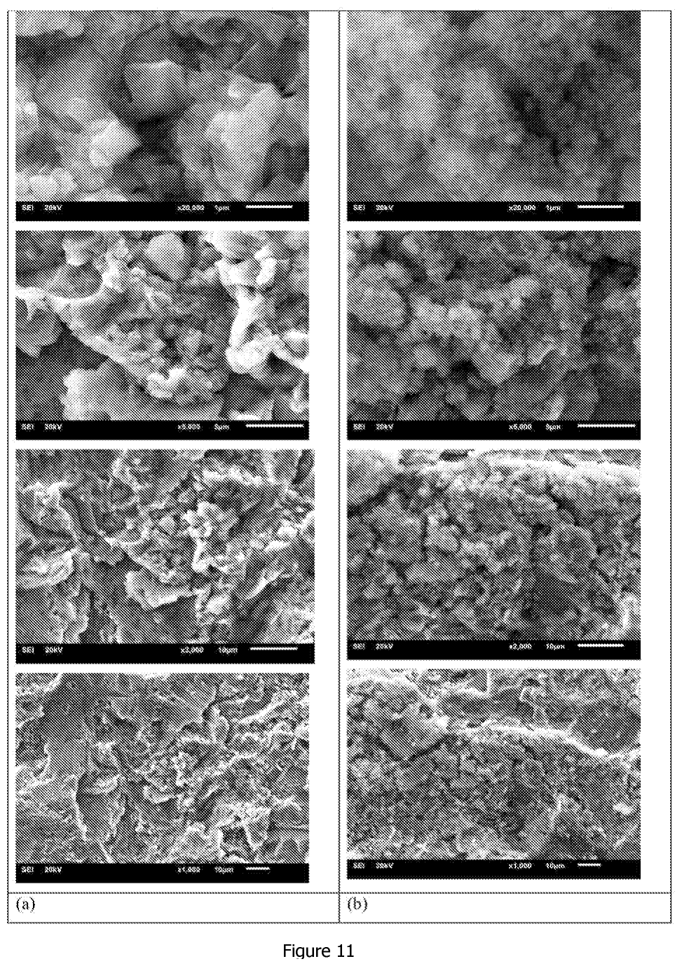

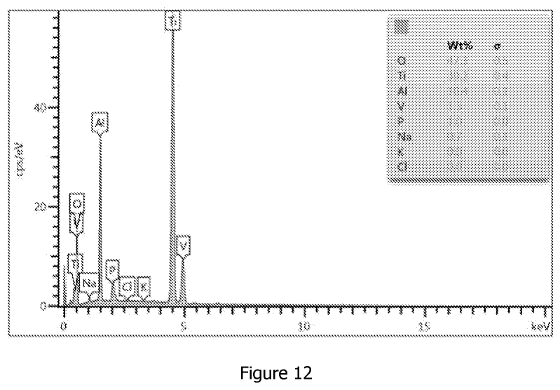

When SEM (FIG. 11) and EDS (FIG. 12) images of control samples (Ti-6Al-4V implant) and dental implant coated with antibacterial MOF/Ag structure (4) suspended in PBS are examined, it is seen that as a result of the reaction of dental implant surface coated with MOF/Ag structure (4) with the phosphate ions (PO₄³⁻) in PBS solution, it is smoother. This condition shows that the prepared implant can easily interact with phosphate ions, which are the main component of the bone, and accelerate bone formation (osseointegration). The dental implant coated with MOF/Ag structure (4) kept in PBS contains 1.0% phosphorus (P) according to the EDS analysis results, while no phosphorus was found in the control samples. Accordingly, a dental implant with high osseointegration capability and biocompatibility is provided with the dental implant of the invention.

Antibacterial examinations of the dental implant coated with antibacterial MOF/Ag structure (4) were performed using Staphylococcus aureus ATCC25923 (S. aureus) and Escherichia coli ATCC25922 (E. coli) bacteria. In this embodiment, the following process steps are followed;
i. Performing cultivation into Nutrient Broth (NB) medium (pH 7.2) from glycerol stock of the bacteria and obtaining bacterial suspension by incubating at 37° C. for 24 hours,
ii. Performing cultivation into NB medium under the same conditions by taking from the colonies here after the solid nutrient agar plates are prepared and subsequently preparing a bacterial suspension with a density of 105 CFU/mL,
iii. Placing each 10 mm×2 mm sized sample in 24-well culture petri dishes after exposing to UV sterilization (0.8 mL bacterial suspension, placed according to Ren et al., 2014) iv. Diluting all samples such that dilution amount is 103 CFU/mL,
v. Taking 100 μL of the treatment suspensions after the incubation and planting in NA plates and incubating the same at 37° C. for 24 hours
vi. Counting the amount of colonies in the petri dishes so as to calculate the effect of the samples on the survival of bacteria.

The samples of dental implants coated with only antibacterial MOF/Ag structure (4) exhibit strong antibacterial effect against S. aureus and E. coli according to obtained results. It was seen that samples without silver (Ag) did not have any antibacterial effect at the end of 24 hours and formed colonies that is beyond measure.

### REFERENCES

1) Abron A., Hopfensperger M., Thompson J., Cooper L. F., 2001. Evaluation of a predictive model for implant surface topography effects on early osseointegration in the rat tibia model. J Prosthet Dent 85, 40-46.
2) Avila, G., Misch, K., Galindo-Moreno, P., Wang, H. L. 2009. “Implant surface treatment using biomimetic agents”, Implant Dentistry, 18 (1), 17-26.
3) Branemark R., Branemark P. I., Rydevik B., Myers R. R., 2001. Osseointegration in skeletal reconstruction and rehabilitation: A review. J Rehabil Res Dev 38, 175-181.
4) Canalis E., Delany A. M., 2002. Mechanisms of glucocorticoid action in bone. Ann N Y Acad Sci, 966, 73-81.
5) De Jonge, L. T., Leeuwenburgh, S. C., Wolke, J. G., Jansen, J. A., 2008. Organic-inorganic surface modifications for titanium implant surfaces. Pharmaceutical Research 25 (10), 2357-2369.
6) Furukawa, H., Cordova, K. E., O'Keeffe, M., Yaghi, O. M. 2013. “The Chemistry and Applications of Metal-Organic Frameworks Science”, 341, 1230444.
7) Horcajada, P., Gref, R., Baati, T., Allan, P. K., Maurin, G., Couvreur, P., Ferey, G., Morris, R. E., Serre, C. 2012. Metal-organic frameworks in biomedicine, Chem. Rev. 112, 1232-1268.
8) Huxford, R. C., Rocca, J. D., Lin, W. 2010. “Metal-organic frameworks as potential drug carriers”, Current Opinion in Chemical Biology, 14:262-268.
9) Liu X., Tian A., You J., Zhang H., Wu L., Bai X., Lei Z., Shi X., Xue X., Wang H. 2016. Antibacterial abilities and biocompatibilities of Ti—Ag alloys with nanotubular coatings. International Journal of Nanomedicine 11, 5743-5755.
10) Lumbikanonda N., Sammons R., 2001. Bone cell attachment to dental 111 implants of different surface characteristics. Int J Oral Maxillofac Implants 16, 627-36.
11) Miao X., Wang D., Xu L., Wang J., Zeng D., Lin S., Huang C., Liu X., Jiang X., 2017. The response of human osteoblasts, epithelial cells, fibroblasts, macrophages and oral bacteria to nanostructured titanium surfaces: a systematic study. Int J Nanomedicine 12, 1415-1430.
12) Mortada, B., Matar, T. A., Sakaya, A., Atallah, H., Ali, Z. K., Karam, P., Hmadeh, M. 2017. “Postmetalated Zirconium Metal-organic frameworks as a Highly Potent Bactericide”, Inorganic Chemistry 56 (8), 4739-4744.
13) Neut D., van de Belt H., van Horn J. R., van der Mei H. C., Busscher H. J., 2003. Residual gentamicin-release from antibiotic-loaded polymethylmethacrylate beads after 5 years of implantation. Biomaterials, 24(10), 1829-1831.
14) Scacchi M., Merz B. R., Schar A. R., 2000. The development of the ITI dental implant system. Part 2: 1998-2000: Steps into the next millennium. Clin Oral Implants Res 11, Suppl 1, 22-32.
15) Souza F. A., Furtado T. S. M., Dayube U. R. C., Melo W. M., Nishioka R. S., Poli P. P., Maiorana C., Carvalho P. S. P., 2019. Comparative in vivo study of alloy titanium implants with two different surfaces: biomechanical and SEM analysis. Clinical Oral Investigations, 23:4383-4397.
16) Tamilvanan S., Venkateshan N., Ludwig A., 2008. The potential of lipid- and polymer-based drug delivery carriers for eradicating biofilm consortia on device-related nosocomial infections. J Control Release, 128 (1):2-22.
17) Yokoyama K., Ichikawa T., Murakami H., Miyamoto Y., Asaoka K., 2002. Fracture mechanisms of retrieved titanium screw thread in dental implants. Biomaterials 23, 2459-2465.

1. An antibacterial/antimicrobial dental implant (1) preparation method that contains a metal-organic framework (MOF) structure involving antibacterial metal ions, especially silver (Ag), thus can release antibacterial metal ions and exhibits a long-term antibacterial/antimicrobial effect, has osseointegration capability and high biocompatibility, characterized in that, it comprises the following process steps;
i. Adding a solution containing 100-900 mg 1,2,4,5-benzentetracarboxylic acid (pyromellitic acid) in 1-500 mL of water on a solution containing 100-900 mg of zirconium tetrachloride (ZrCl₄) in 1-500 mL of water and stirring the same for 1-600 min,
ii. Adding 1-50 mL of hydrochloric acid (HCl) (37%, w/w) to each container and keeping the resulting solution for 0.1-96 hours in the incubator at 40-220° C., after dispersing 1-100 mL of the solution into Teflon containers,
iii. Taking the formed framework structure from the incubator at the end of 0.1-96 hours and filtering the same under vacuum and washing the same with hot water,
iv. Heating the framework structure at 30-180° C. for 0.1-48 hours so as to remove the solvent molecules in its pores,
v. Adding 1-1000 mg of framework structure to 1-1000 mL of methanol (MeOH) containing 1-100 mg of silver nitrate (AgNO₃) and mixing the same for 0.1-48 hours at 25-150° C. so as to obtain a MOF/Ag structure (4),
vi. Separating the obtained MOF/Ag structure (4) from the solution and washing the same with methanol (MeOH),
vii. Drying MOF/Ag structure (4) at 30-180° C. under vacuum,
viii. Loading the obtained MOF/Ag structure (4) into the cavity (3) formed in the infrastructure (5) section of the dental implant.

2. A dental implant prepared by a method according to claim 1.

3. A dental implant according to claim 2, characterized in that; it comprises porous metal-organic framework (MOF) structure (4) that is placed in the cavity (3) section formed in the infrastructure (5) section, loaded with drugs, antibiotics, antimicrobial/antibacterial agents, provides the release of drug, antibiotic, antimicrobial/antibacterial agent release therein depending on the temperature increase of the tissue in contact and micro/nano channels (9) that are formed by drilling 100 nm-500 μg diameter holes at different angles on the implant, provides a passage for the antimicrobial/antibacterial agent release from MOF/Ag to the tissue.

4. A method for preparing a dental implant that is coated with metal-organic framework (MOF) structure containing antibacterial metal ions, especially silver (Ag), thus can release long-term and controlled antibacterial metal ions and exhibits a long-term antibacterial/antimicrobial effect, has osseointegration capability and high biocompatibility, wherein the adverse effects of vanadium (V) on human health are eliminated by preventing the release of vanadium (V) ions from the basic implant layer to the tissue, characterized in that, it comprises the following process steps;
i. Roughening the surface of Ti-6Al-4V implants under 2-8 bar pressure for 50 seconds by bombarding with Al₂O₃ particles having 300-425 μm dimension,
ii. Oxidizing the surface of rough Ti-6Al-4V implants at 450-700° C. using a tube furnace,
iii. Adding a solution containing 100-900 mg 1,2,4,5-benzentetracarboxylic acid (pyromellitic acid) in 1-500 mL of water on a solution containing 100-900 mg of zirconium tetrachloride (ZrCl₄) in 1-500 mL of water and stirring the same for 1-600 min,
iv. Adding 1-50 mL of hydrochloric acid (HCl) (37%, w/w) to each container and keeping the resulting solution for 0.1-96 hours in the incubator at 40-220° C., after dispersing 1-100 mL of the solution into Teflon containers,
v. Taking the formed framework structure from the incubator at the end of 0.1-96 hours and filtering the same under vacuum and washing the same with hot water,
vi. Heating the framework structure at 30-180° C. for 0.1-48 hours so as to remove the solvent molecules in its pores,
i. Adding 1-1000 mg of framework structure to 1-1000 mL of methanol (MeOH) containing 1-100 mg of silver nitrate (AgNO₃) and mixing the same for 0.1-48 hours at 25-150° C. so as to obtain a MOF/Ag structure (4),
ii. Separating the obtained MOF/Ag structure (4) from the solution and washing the same with methanol (MeOH),
ix. Drying MOF/Ag structure (4) at 30-180° C. under vacuum,
x. Adding the dried MOF/Ag structure (4) in 10 ml of ethyl alcohol and preparing the same as a suspension by means of an ultrasonic bath,
xi. Coating the rough Ti-6Al-4V implants whose surface obtained in the process step (ii) is oxidized, with the MOF/Ag structure (4) converted into a suspension and is dried at 80-180° C. temperature by means of dipping method,
i. Continuing the coating process until all of the suspended MOF/Ag structure (4) of the process step (xi) is consumed.

5. A method according to claim 4, characterized in that; it comprises the following process steps;
i. Roughening the surface of Ti-6Al-4V implants under 4 bar pressure for 50 seconds by bombarding with Al₂O₃ particles having 300-425 μm dimension,
ii. Oxidizing the surface of rough Ti-6Al-4V implants at 600° C. using a tube furnace,
iii. Adding a solution containing 100-900 mg 1,2,4,5-benzentetracarboxylic acid (pyromellitic acid) in 1-500 mL of water on a solution containing 100-900 mg of zirconium tetrachloride (ZrCl₄) in 1-500 mL of water and stirring the same for 1-600 min,
iv. Adding 1-50 mL of hydrochloric acid (HCl) (37%, w/w) to each container and keeping the resulting solution for 0.1-96 hours in the incubator at 40-220° C., after dispersing 1-100 mL of the solution into Teflon containers,
v. Taking the formed framework structure from the incubator at the end of 0.1-96 hours and filtering the same under vacuum and washing the same with hot water,
vi. Heating the framework structure at 30-180° C. for 0.1-48 hours so as to remove the solvent molecules in its pores,
vii. Adding 1-1000 mg of framework structure to 1-1000 mL of methanol (MeOH) containing 1-100 mg of silver nitrate (AgNO₃) and mixing the same for 0.1-48 hours at 25-150° C. so as to obtain a MOF/Ag structure (4),
viii. Separating the obtained MOF/Ag structure (4) from the solution and washing the same with methanol (MeOH),
ix. Drying MOF/Ag structure (4) at 30-180° C. under vacuum,
x. Adding the dried MOF/Ag structure (4) in 10 ml of alcohol and preparing the same as a suspension by means of an ultrasonic bath,
xi. Coating the rough Ti-6Al-4V implants whose surface obtained in the process step (ii) is oxidized, with the MOF/Ag structure (4) converted into a suspension and is dried at 140° C. temperature by means of dipping method,
xii. Continuing the coating process until all of the suspended MOF/Ag structure (4) of the process step (xi) is consumed.

6. A dental implant prepared with a method according to claim 4.

7. A dental implant according to claim 6, characterized in that; it comprises Ti-6Al-4V coated with a metal-organic framework structure MOF/Ag containing silver (Ag) that provides long-term and controlled silver (Ag) release and at the same time prevents the release of vanadium (V) ions from the basic implant layer containing Ti-6Al-4V alloy, has high osseointegration ability and biocompatibility.

8. A dental implant according to claim 7, characterized in that; Ti-6Al-4V alloy surface has a rough and oxidized structure.

9. A dental implant prepared with a method according to claim 5.
